(19) United States
(12) Patent Application Publication
(10) Pub. No.: US 2011/0257707 A1
(43) Pub. Date: Oct. 20, 2011

(54) NEUROSTIMULATION SYSTEM AND METHOD WITH ADJUSTABLE PROGRAMMING RATE

(75) Inventor: Sridhar Kothandaraman, Valencia, CA (US)

(73) Assignee: BOSTON SCIENTIFIC NEUROMODULATION CORPORATION, Valencia, CA (US)

(21) Appl. No.: 13/090,073

(22) Filed: Apr. 19, 2011

Related U.S. Application Data
(60) Provisional application No. 61/325,736, filed on Apr. 19, 2010.

(51) Int. Cl. A61N 1/36

(52) U.S. Cl. 607/60

(57) ABSTRACT

Neurostimulation systems, control systems, and methods for providing therapy to a patient are provided. Electrical stimulation energy is delivered to a tissue region in accordance with different stimulation parameter sets. The delivered electrical stimulation energy is incrementally transitioned through a first series of the different stimulation parameter sets at a user-defined rate in response to a single user actuation of a control mechanism. The user-defined rate is adjusted, and the delivered electrical stimulation energy is incrementally transitioned through a second series of the different stimulation parameter sets at the adjusted rate in response to a single user actuation of the control mechanism.

### RELATED APPLICATION

The present application claims the benefit under 35 U.S.C. §119 to U.S. provisional patent application Ser. No. 61/325,736, filed Apr. 19, 2010. The foregoing application is hereby incorporated by reference into the present application in its entirety.

### FIELD OF THE INVENTION

The present inventions relate to tissue stimulation systems, and more particularly, to neurostimulation systems for programming neurostimulation leads.

### BACKGROUND OF THE INVENTION

Implantable neurostimulation systems have proven therapeutic in a wide variety of diseases and disorders. Pacemakers and Implantable Cardiac Defibrillators (ICDs) have proven highly effective in the treatment of a number of cardiac conditions (e.g., arrhythmias). Spinal Cord Stimulation (SCS) systems have long been accepted as a therapeutic modality for the treatment of chronic pain syndromes, and the application of tissue stimulation has begun to expand to additional applications such as angina pectoralis and incontinence. Deep Brain Stimulation (DBS) has also been applied therapeutically for well over a decade for the treatment of refractory chronic pain syndromes, and DBS has also recently been applied in additional areas such as movement disorders and epilepsy. Further, in recent investigations, Peripheral Nerve Stimulation (PNS) systems have demonstrated efficacy in the treatment of chronic pain syndromes and incontinence, and a number of additional applications are currently under investigation. Furthermore, Functional Electrical Stimulation (FES) systems, such as the Freehand system by NeuroControl (Cleveland, Ohio), have been applied to restore some functionality to paralyzed extremities in spinal cord injury patients.

These implantable neurostimulation systems typically include one or more electrode carrying stimulation leads, which are implanted at the desired stimulation site, and a neurostimulator (e.g., an implantable pulse generator (IPG)) implanted remotely from the stimulation site, but coupled either directly to the stimulation lead(s) or indirectly to the stimulation lead(s) via a lead extension. The neurostimulation system may further comprise an external control device to remotely instruct the neurostimulator to generate electrical stimulation pulses in accordance with selected stimulation parameters.

Electrical stimulation energy may be delivered from the neurostimulator to the electrodes in the form of an electrical pulsed waveform. Thus, stimulation energy may be controllably delivered to the electrodes to stimulate neural tissue. The combination of electrodes used to deliver electrical pulses to the targeted tissue constitutes an electrode combination, with the electrodes capable of being selectively programmed to act as anodes (positive), cathodes (negative), or left off (zero). In other words, an electrode combination represents the polarity being positive, negative, or zero. Other parameters that may be controlled or varied include the amplitude, width, and rate of the electrical pulses provided through the electrode array. Each electrode combination, along with the electrical pulse parameters, can be referred to as a “stimulation parameter set.”

With some neurostimulation systems, and in particular, those with independently controlled current or voltage sources, the distribution of the current to the electrodes (including the case of the neurostimulator, which may act as an electrode) may be varied such that the current is supplied via numerous different electrode configurations. In different configurations, the electrodes may provide current or voltage in different relative percentages of positive and negative current or voltage to create different electrical current distributions (i.e., fractionalized electrode combinations).

As briefly discussed above, an external control device can be used to instruct the neurostimulator to generate electrical stimulation pulses in accordance with the selected stimulation parameters. Typically, the stimulation parameters programmed into the neurostimulator can be adjusted by manipulating controls on the external control device to modify the electrical stimulation provided by the neurostimulator system to the patient. Thus, in accordance with the stimulation parameters programmed by the external control device, electrical pulses can be delivered from the neurostimulator to the stimulation electrode(s) to stimulate or activate a volume of tissue in accordance with a set of stimulation parameters and provide the desired efficacious therapy to the patient. The best stimulus parameter set will typically be one that delivers stimulation energy to the volume of tissue that must be stimulated in order to provide the therapeutic benefit (e.g., treatment of pain), while minimizing the volume of non-target tissue that is stimulated.

However, the number of electrodes available, combined with the ability to generate a variety of complex stimulation pulses, presents a huge selection of stimulation parameter sets to the clinician or patient. For example, if the neurostimulation system to be programmed has an array of sixteen electrodes, millions of stimulation parameter sets may be available for programming into the neurostimulation system. Today, neurostimulation system may have up to thirty-two electrodes, thereby exponentially increasing the number of stimulation parameters sets available for programming.

To facilitate such selection, the clinician generally programs the neurostimulator through a computerized programming system. This programming system can be a self-contained hardware/software system, or can be defined predominantly by software running on a standard personal computer (PC). The PC or custom hardware may actively control the characteristics of the electrical stimulation generated by the neurostimulator to allow the optimum stimulation parameters to be determined based on patient feedback or other means and to subsequently program the neurostimulator with the optimum stimulation parameter set or sets, which will typically be those that stimulate all of the target tissue in order to provide the therapeutic benefit, yet minimizes the volume of non-target tissue that is stimulated. The computerized programming system may be operated by a clinician attending the patient in several scenarios.

For example, in order to achieve an effective result from SCS, the lead or leads must be placed in a location, such that the electrical stimulation will cause paresthesia. The paresthesia induced by the stimulation and perceived by the patient should be located in approximately the same place in the patient's body as the pain that is the target of treatment. If a lead is not correctly positioned, it is possible that the patient will receive little or no benefit from an implanted SCS system. Thus, correct lead placement can mean the difference between effective and ineffective pain therapy. When electrical leads are implanted within the patient, the computerized programming system, in the context of an operating room (OR) mapping procedure, may be used to instruct the neurostimulator to apply electrical stimulation to test placement of the leads and/or electrodes, thereby assuring that the leads and/or electrodes are implanted in effective locations within the patient.

Once the leads are correctly positioned, a fitting procedure, which may be referred to as a navigation session, may be performed using the computerized programming system to program the external control device, and if applicable the neurostimulator, with a set of stimulation parameters that best addresses the painful site. Thus, the navigation session may be used to pinpoint the stimulation region or areas correlating to the pain. Such programming ability is particularly advantageous for targeting the tissue during implantation, or after implantation should the leads gradually or unexpectedly move that would otherwise relocate the stimulation energy away from the target site. By reprogramming the neurostimulator (typically by independently varying the stimulation energy on the electrodes), the stimulation region can often be moved back to the effective pain site without having to re-operate on the patient in order to reposition the lead and its electrode array. When adjusting the stimulation region relative to the tissue, it is desirable to make small changes in the proportions of current, so that changes in the spatial recruitment of nerve fibers will be perceived by the patient as being smooth and continuous and to have incremental targeting capability.

One known computerized programming system for SCS is called the Bionic Navigator®, available from Boston Scientific Neuromodulation, Sylmar, Calif. The Bionic Navigator® is a software package that operates on a suitable PC and allows clinicians to program stimulation parameters into an external handheld programmer (referred to as a remote control). Each set of stimulation parameters, including fractionalized current distribution to the electrodes (as percentage cathodic current, percentage anodic current, or off), programmed by the Bionic Navigator® may be stored in both the Bionic Navigator® and the remote control and combined into a stimulation program that can then be used to stimulate multiple regions within the patient.

Prior to creating the stimulation programs, the Bionic Navigator® may be operated by a clinician in a “manual mode” to manually select the percentage cathodic current and percentage anodic current flowing through the electrodes, or may be operated by the clinician in a “navigation mode” to electrically “steer” the current along the implanted leads in real-time, thereby allowing the clinician to determine the most efficacious stimulation parameter sets that can then be stored and eventually combined into stimulation programs. In the navigation mode, the Bionic Navigator® can store selected fractionalized electrode combinations that can be displayed to the clinician as marks representing corresponding stimulation regions relative to the electrode array.

The Bionic Navigator® performs current steering in accordance with a steering or navigation table. For example, as shown in Appendix A, an exemplary navigation table, which includes a series of reference electrode combinations (for a lead of 8 electrodes) with associated fractionalized current values (i.e., fractionalized electrode combinations), can be used to gradually steer electrical current from one basic electrode combination to the next, thereby electronically steering the stimulation region along the leads. The marks can then be created from selected fractionalized electrode combinations within the navigation table that can be combined with the electrical pulse parameters to create one or more stimulation programs.

For example, the navigation table can be used to gradually steer current between a basic electrode combination consisting of a cathodic electrode 3 and an anodic electrode 5 (represented by stimulation set 161) and either a basic electrode combination consisting of a cathodic electrode 3 and an anodic electrode 1 (represented by stimulation set 141) or a basic electrode combination consisting of a cathodic electrode 3 and an anodic electrode 6 (represented by stimulation set 181). That is, electrical current can be incrementally shifted from anodic electrode 5 to the anodic electrode 1 as one steps upward through the navigation table from stimulation set 161 to stimulation set 141, and from anodic electrode 5 to anodic electrode 6 as one steps downward through the navigation table from stimulation set 161 to stimulation set 181.

Despite the fact that computerized programming systems have been used to speed up the programming process, programming of an electrical stimulation system using present-day computerized programming systems may still be a relatively time-consuming process. In particular, because computerized programming systems have uniform rates at which the stimulation parameter sets are modified, the speed at which the stimulation parameter sets are tested to achieve optimal stimulation therapy may not match the desired programming speed. For example, different patients have different perception threshold times (the time between the change in the applied stimulation energy and patient feeling the change in the applied stimulation energy). If a patient has a perception threshold time that is not quick enough to feel the changes in the stimulation parameters, otherwise optimal stimulation parameter sets may not be adequately tested, and therefore, will not be programmed in the neurostimulation system. In contrast, if the patient has a perception threshold time that is significantly quicker than the rate at which the stimulation parameter sets are changed by the computerized programming system, programming of the neurostimulation system may become too tedious, and thus, optimal stimulation parameters sets may not ever be tested at all.

There, thus, remains a need for a computerized programming system that is capable of more efficiently programming a neurostimulation system.

### SUMMARY OF THE INVENTION

In accordance with a first aspect of the present inventions, a neurostimulation system is provided. The neurostimulation system comprises a neurostimulator configured for delivering electrical stimulation energy to a tissue region in accordance with different stimulation parameter sets, which may, e.g., contain different active electrode combinations, such as fractionalized electrode combinations, or may contain different values for at least one of a pulse amplitude, a pulse width, and a pulse rate.

The neurostimulation system further comprises an external control device configured for, in response to a single user actuation of a control mechanism (e.g., a computer icon), instructing the neurostimulator to incrementally transition the delivered electrical stimulation energy through the different stimulation parameter sets at a user-adjustable rate. In one embodiment, the user-adjustable rate is a function of the inverse of a time interval between immediately adjacent incremental transitions of the delivered electrical stimulation energy. In another embodiment, the user-adjustable rate is a function of a magnitude difference between immediately adjacent stimulation parameter values within the different stimulation parameter sets. In an optional embodiment, the external control device is further configured for programming at least one of the stimulation parameter sets into the neurostimulator.

In accordance with a second aspect of the present inventions, a neurostimulation control system for a neurostimulator that conveys electrical stimulation energy to a tissue region in accordance with different electrical stimulation parameter sets is provided. The control system comprises a control mechanism (e.g., a computer icon) configured for being actuated by a user, and at least one processor configured for generating the different stimulation parameter sets (which may, e.g., be any of those described above) in response to a single user actuation of the control mechanism. The control system further comprises telemetry circuitry configured for transmitting the different stimulation parameters to the neurostimulator, such that the neurostimulator incrementally transitions the delivered electrical stimulation energy through the different stimulation parameter sets at defined rate (which may, e.g., be any of the rates described above), and a user interface configured for allowing a user to adjust the defined rate.

In accordance with a third aspect of the present inventions, a method of providing therapy to a patient is provided. The method comprises delivering electrical stimulation energy to a tissue region in accordance with different stimulation parameter sets (which may, e.g., be any of those described above), incrementally transitioning the delivered electrical stimulation energy through a first series of the different stimulation parameter sets at a user-defined rate (which may, e.g., be any of the rates described above) in response to a single user actuation of a control mechanism (e.g., a computer icon), adjusting the user-defined rate, and incrementally transitioning the delivered electrical stimulation energy through a second series of the different stimulation parameter sets at the adjusted rate in response to a single user actuation of the control mechanism. An optional method comprises programming the at least one of the second series of stimulation parameter sets into the memory of a neurostimulation device.

Other and further aspects and features of the invention will be evident from reading the following detailed description of the preferred embodiments, which are intended to illustrate, not limit, the invention.

### BRIEF DESCRIPTION OF THE DRAWINGS

The drawings illustrate the design and utility of preferred embodiments of the present invention, in which similar elements are referred to by common reference numerals. In order to better appreciate how the above-recited and other advantages and objects of the present inventions are obtained, a more particular description of the present inventions briefly described above will be rendered by reference to specific embodiments thereof, which are illustrated in the accompanying drawings. Understanding that these drawings depict only typical embodiments of the invention and are not therefore to be considered limiting of its scope, the invention will be described and explained with additional specificity and detail through the use of the accompanying drawings in which:

FIG. 1 is perspective view of one embodiment of a SCS system arranged in accordance with the present inventions;

FIG. 2 is a plan view of the SCS system of FIG. 1 in use with a patient;

FIG. 3 is a side view of an implantable pulse generator and a pair of stimulation leads that can be used in the SCS system of FIG. 1;

FIG. 4 is a plan view of a remote control that can be used in the SCS system of FIG. 1;

FIG. 5 is a block diagram of the internal componentry of the remote control of FIG. 4;

FIG. 6 is a block diagram of the components of a clinician programmer that can be used in the SCS system of FIG. 1;

FIG. 7 is a first operating room mapping screen that can be displayed by the clinician programmer of FIG. 1;

FIG. 8 is a second operating room mapping screen that can be displayed by the clinician programmer of FIG. 1, particularly showing a first fractionalized electrode configuration in the E-Troll mode;

FIG. 9 is a third operating room mapping screen that can be displayed by the clinician programmer of FIG. 1, particularly showing a second fractionalized electrode configuration in the E-troll mode;

FIG. 10 is a fourth operating room mapping screen that can be displayed by the clinician programmer of FIG. 1, particularly showing a third fractionalized electrode configuration in the E-troll mode;

FIG. 11 is a first navigator programming screen that can be displayed by the clinician programmer of FIG. 1;

FIG. 12 is a second navigator programming screen that can be displayed by the clinician programmer of FIG. 1, particularly showing a fractionalized electrode configuration; and

FIG. 13 is a third navigator programming screen that can be displayed by the clinician programmer of FIG. 1, particularly showing the creation of four marks and corresponding stimulation regions.

### DETAILED DESCRIPTION OF THE EMBODIMENTS

The description that follows relates to a spinal cord stimulation (SCS) system. However, it is to be understood that while the invention lends itself well to applications in SCS, the invention, in its broadest aspects, may not be so limited. Rather, the invention may be used with any type of implantable electrical circuitry used to stimulate tissue. For example, the present invention may be used as part of a pacemaker, a defibrillator, a cochlear stimulator, a retinal stimulator, a stimulator configured to produce coordinated limb movement, a cortical stimulator, a deep brain stimulator, peripheral nerve stimulator, microstimulator, or in any other neurostimulator configured to treat urinary incontinence, sleep apnea, shoulder sublaxation, headache, etc.

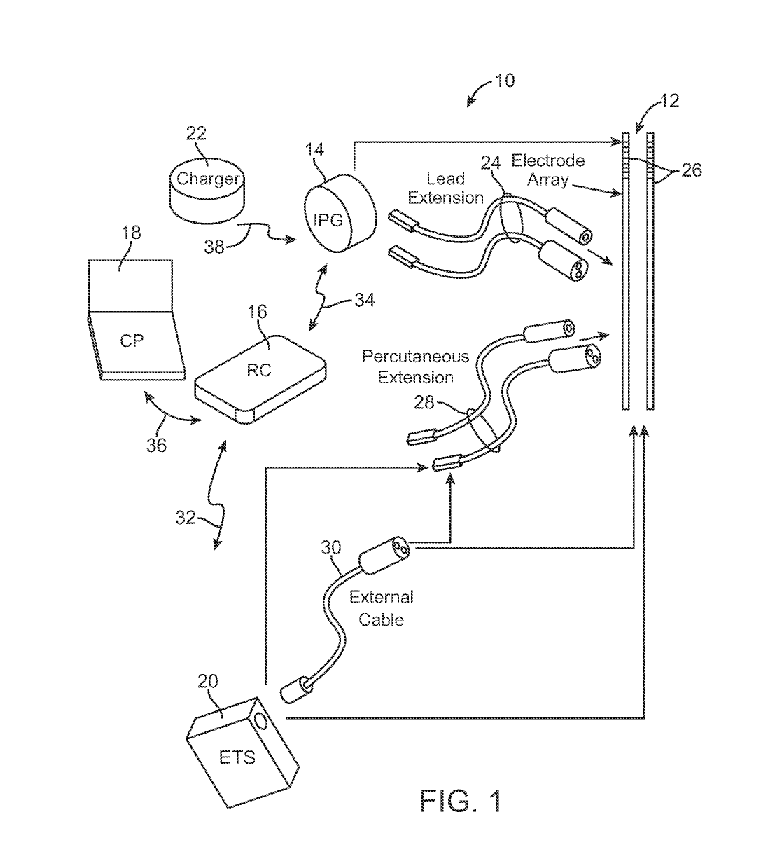

Turning first to FIG. 1, an exemplary SCS system 10 generally includes a plurality (in this case, two) of implantable neurostimulation leads 12, an implantable pulse generator (IPG) 14, an external remote controller RC 16, a clinician's programmer (CP) 18, an external trial stimulator (ETS) 20, and an external charger 22.

The IPG 14 is physically connected via one or more percutaneous lead extensions 24 to the neurostimulation leads 12, which carry a plurality of electrodes 26 arranged in an array. In the illustrated embodiment, the neurostimulation leads 12 are percutaneous leads, and to this end, the electrodes 26 are arranged in-line along the neurostimulation leads 12. As will be described in further detail below, the IPG 14 includes pulse generation circuitry that delivers electrical stimulation energy in the form of a pulsed electrical waveform (i.e., a temporal series of electrical pulses) to the electrode array 26 in accordance with a set of stimulation parameters.

The ETS 20 may also be physically connected via the percutaneous lead extensions 28 and external cable 30 to the neurostimulation leads 12. The ETS 20, which has similar pulse generation circuitry as the IPG 14, also delivers electrical stimulation energy in the form of a pulse electrical waveform to the electrode array 26 accordance with a set of stimulation parameters. The major difference between the ETS 20 and the IPG 14 is that the ETS 20 is a non-implantable device that is used on a trial basis after the neurostimulation leads 12 have been implanted and prior to implantation of the IPG 14, to test the responsiveness of the stimulation that is to be provided. Further details of an exemplary ETS are described in U.S. Pat. No. 6,895,280, which is expressly incorporated herein by reference.

The RC 16 may be used to telemetrically control the ETS 20 via a bi-directional RF communications link 32. Once the IPG 14 and neurostimulation leads 12 are implanted, the RC 16 may be used to telemetrically control the IPG 14 via a bi-directional RF communications link 34. Such control allows the IPG 14 to be turned on or off and to be programmed with different stimulation parameter sets. The IPG 14 may also be operated to modify the programmed stimulation parameters to actively control the characteristics of the electrical stimulation energy output by the IPG 14. As will be described in further detail below, the CP 18 provides clinician detailed stimulation parameters for programming the IPG 14 and ETS 20 in the operating room and in follow-up sessions.

The CP 18 may perform this function by indirectly communicating with the IPG 14 or ETS 20, through the RC 16, via an IR communications link 36. Alternatively, the CP 18 may directly communicate with the IPG 14 or ETS 20 via an RF communications link (not shown). The clinician detailed stimulation parameters provided by the CP 18 are also used to program the RC 16, so that the stimulation parameters can be subsequently modified by operation of the RC 16 in a stand-alone mode (i.e., without the assistance of the CP 18).

The external charger 22 is a portable device used to transcutaneously charge the IPG 14 via an inductive link 38. For purposes of brevity, the details of the external charger 22 will not be described herein. Details of exemplary embodiments of external chargers are disclosed in U.S. Pat. No. 6,895,280, which has been previously incorporated herein by reference. Once the IPG 14 has been programmed, and its power source has been charged by the external charger 22 or otherwise replenished, the IPG 14 may function as programmed without the RC 16 or CP 18 being present.

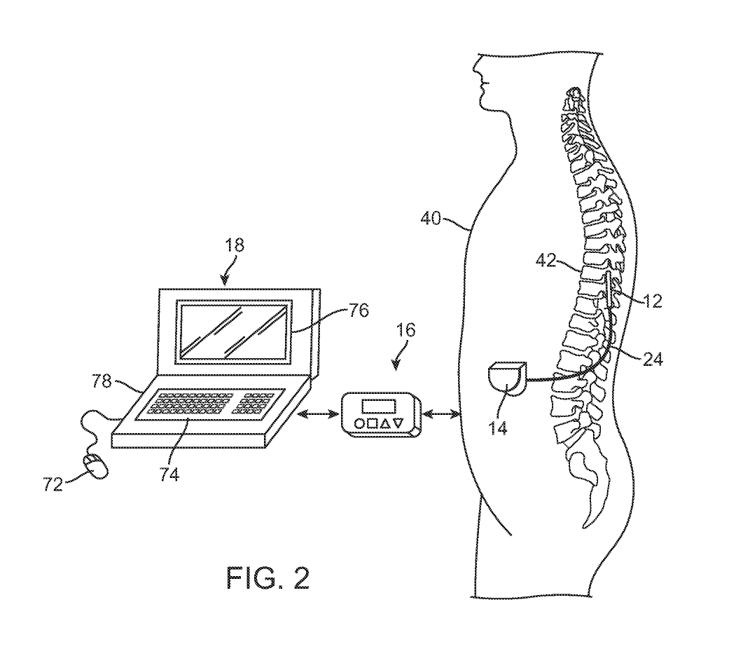

As shown in FIG. 2, the electrode leads 12 are implanted within the spinal column 42 of a patient 40. The preferred placement of the electrode leads 12 is adjacent, i.e., resting upon, the spinal cord area to be stimulated. Due to the lack of space near the location where the electrode leads 12 exit the spinal column 42, the IPG 14 is generally implanted in a surgically-made pocket either in the abdomen or above the buttocks. The IPG 14 may, of course, also be implanted in other locations of the patient's body. The lead extensions 24 facilitate locating the IPG 14 away from the exit point of the electrode leads 12. As there shown, the CP 18 communicates with the IPG 14 via the RC 16.

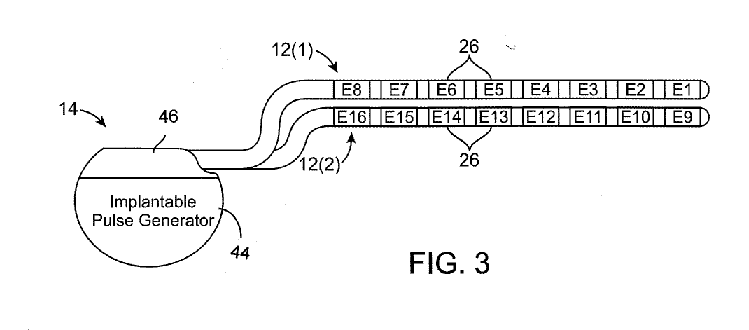

Referring now to FIG. 3, the external features of the neurostimulation leads 12 and the IPG 14 will be briefly described. One of the neurostimulation leads 12(1) has eight electrodes 26 (labeled E1-E8), and the other neurostimulation lead 12(2) has eight electrodes 26 (labeled E9-E16). The actual number and shape of leads and electrodes will, of course, vary according to the intended application. The IPG 14 comprises an outer case 44 for housing the electronic and other components (described in further detail below), and a connector 46 to which the proximal ends of the neurostimulation leads 12 mates in a manner that electrically couples the electrodes 26 to the electronics within the outer case 44. The outer case 44 is composed of an electrically conductive, biocompatible material, such as titanium, and forms a hermetically sealed compartment wherein the internal electronics are protected from the body tissue and fluids. In some cases, the outer case 44 may serve as an electrode.

The IPG 14 includes a battery and pulse generation circuitry that delivers the electrical stimulation energy in the form of a pulsed electrical waveform to the electrode array 26 in accordance with a set of stimulation parameters programmed into the IPG 14. Such stimulation parameters may comprise electrode combinations, which define the electrodes that are activated as anodes (positive), cathodes (negative), and turned off (zero), percentage of stimulation energy assigned to each electrode (fractionalized electrode combinations), and electrical pulse parameters, which define the pulse amplitude (measured in milliamps or volts depending on whether the IPG 14 supplies constant current or constant voltage to the electrode array 26), pulse width (measured in microseconds), and pulse rate (measured in pulses per second).

Electrical stimulation will occur between two (or more) activated electrodes, one of which may be the IPG case. Simulation energy may be transmitted to the tissue in a monopolar or multipolar (e.g., bipolar, tripolar, etc.) fashion. Monopolar stimulation occurs when a selected one of the lead electrodes 26 is activated along with the case 44 of the IPG 14, so that stimulation energy is transmitted between the selected electrode 26 and case. Bipolar stimulation occurs when two of the lead electrodes 26 are activated as anode and cathode, so that stimulation energy is transmitted between the selected electrodes 26. For example, electrode E3 on the first lead 12 may be activated as an anode at the same time that electrode E11 on the second lead 12 is activated as a cathode. Tripolar stimulation occurs when three of the lead electrodes 26 are activated, two as anodes and the remaining one as a cathode, or two as cathodes and the remaining one as an anode. For example, electrodes E4 and E5 on the first lead 12 may be activated as anodes at the same time that electrode E12 on the second lead 12 is activated as a cathode.

In the illustrated embodiment, IPG 14 can individually control the magnitude of electrical current flowing through each of the electrodes. In this case, it is preferred to have a current generator, wherein individual current-regulated amplitudes from independent current sources for each electrode may be selectively generated. Although this system is optimal to take advantage of the invention, other stimulators that may be used with the invention include stimulators having voltage regulated outputs. While individually programmable electrode amplitudes are optimal to achieve fine control, a single output source switched across electrodes may also be used, although with less fine control in programming. Mixed current and voltage regulated devices may also be used with the invention. Further details discussing the detailed structure and function of IPGs are described more fully in U.S. Pat. Nos. 6,516,227 and 6,993,384, which are expressly incorporated herein by reference.

It should be noted that rather than an IPG, the SCS system 10 may alternatively utilize an implantable receiver-stimulator (not shown) connected to the neurostimulation leads 12. In this case, the power source, e.g., a battery, for powering the implanted receiver, as well as control circuitry to command the receiver-stimulator, will be contained in an external controller inductively coupled to the receiver-stimulator via an electromagnetic link. Data/power signals are transcutaneously coupled from a cable-connected transmission coil placed over the implanted receiver-stimulator. The implanted receiver-stimulator receives the signal and generates the stimulation in accordance with the control signals.

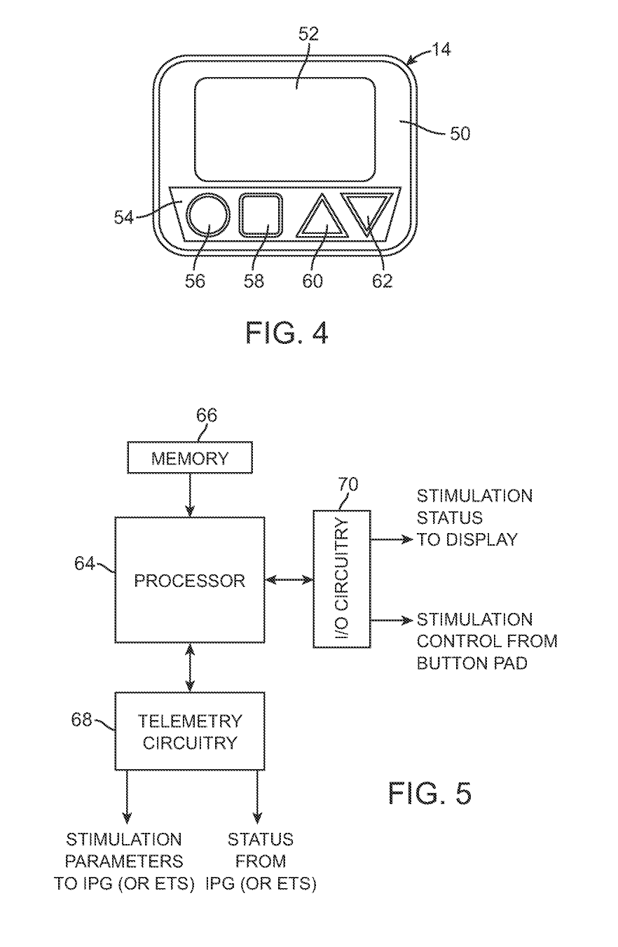

Referring now to FIG. 4, one exemplary embodiment of an RC 16 will now be described. As previously discussed, the RC 16 is capable of communicating with the IPG 14, CP 18, or ETS 20. The RC 16 comprises a casing 50, which houses internal componentry (including a printed circuit board (PCB)), and a lighted display screen 52 and button pad 54 carried by the exterior of the casing 50. In the illustrated embodiment, the display screen 52 is a lighted flat panel display screen, and the button pad 54 comprises a membrane switch with metal domes positioned over a flex circuit, and a keypad connector connected directly to a PCB. In an optional embodiment, the display screen 52 has touchscreen capabilities. The button pad 54 includes a multitude of buttons 56, 58, 60, and 62, which allow the IPG 14 to be turned ON and OFF, provide for the adjustment or setting of stimulation parameters within the IPG 14, and provide for selection between screens.

In the illustrated embodiment, the button 56 serves as an ON/OFF button that can be actuated to turn the IPG 14 ON and OFF. The button 58 serves as a select button that allows the RC 16 to switch between screen displays and/or parameters. The buttons 60 and 62 serve as up/down buttons that can be actuated to increment or decrement any of stimulation parameters of the pulse generated by the IPG 14, including pulse amplitude, pulse width, and pulse rate. For example, the selection button 58 can be actuated to place the RC 16 in a “Pulse Amplitude Adjustment Mode,” during which the pulse amplitude can be adjusted via the up/down buttons 60, 62, a “Pulse Width Adjustment Mode,” during which the pulse width can be adjusted via the up/down buttons 60, 62, and a “Pulse Rate Adjustment Mode,” during which the pulse rate can be adjusted via the up/down buttons 60, 62. Alternatively, dedicated up/down buttons can be provided for each stimulation parameter. Rather than using up/down buttons, any other type of actuator, such as a dial, slider bar, or keypad, can be used to increment or decrement the stimulation parameters. Further details of the functionality and internal componentry of the RC 16 are disclosed in U.S. Pat. No. 6,895,280, which has previously been incorporated herein by reference.

Referring to FIG. 5, the internal components of an exemplary RC 16 will now be described. The RC 16 generally includes a processor 64 (e.g., a microcontroller), memory 66 that stores an operating program for execution by the processor 64, as well as stimulation parameter sets in a navigation table (described below), input/output circuitry, and in particular, telemetry circuitry 68 for outputting stimulation parameters to the IPG 14 and receiving status information from the IPG 14, and input/output circuitry 70 for receiving stimulation control signals from the button pad 54 and transmitting status information to the display screen 52 (shown in FIG. 4). As well as controlling other functions of the RC 16, which will not be described herein for purposes of brevity, the processor 64 generates new stimulation parameter sets in response to the user operation of the button pad 54. These new stimulation parameter sets would then be transmitted to the IPG 14 (or ETS 20) via the telemetry circuitry 68. Further details of the functionality and internal componentry of the RC 16 are disclosed in U.S. Pat. No. 6,895,280, which has previously been incorporated herein by reference.

As briefly discussed above, the CP 18 greatly simplifies the programming of multiple electrode combinations, allowing the user (e.g., the physician or clinician) to readily determine the desired stimulation parameters to be programmed into the IPG 14, as well as the RC 16. Thus, modification of the stimulation parameters in the programmable memory of the IPG 14 after implantation is performed by a user using the CP 18, which can directly communicate with the IPG 14 or indirectly communicate with the IPG 14 via the RC 16. That is, the CP 18 can be used by the user to modify operating parameters of the electrode array 26 near the spinal cord.

As shown in FIG. 2, the overall appearance of the CP 18 is that of a laptop personal computer (PC), and in fact, may be implemented using a PC that has been appropriately configured to include a directional-programming device and programmed to perform the functions described herein. Thus, the programming methodologies can be performed by executing software instructions contained within the CP 18. Alternatively, such programming methodologies can be performed using firmware or hardware. In any event, the CP 18 may actively control the characteristics of the electrical stimulation generated by the IPG 14 (or ETS 20) to allow the optimum stimulation parameters to be determined based on patient feedback and for subsequently programming the IPG 14 (or ETS 20) with the optimum stimulation parameters.

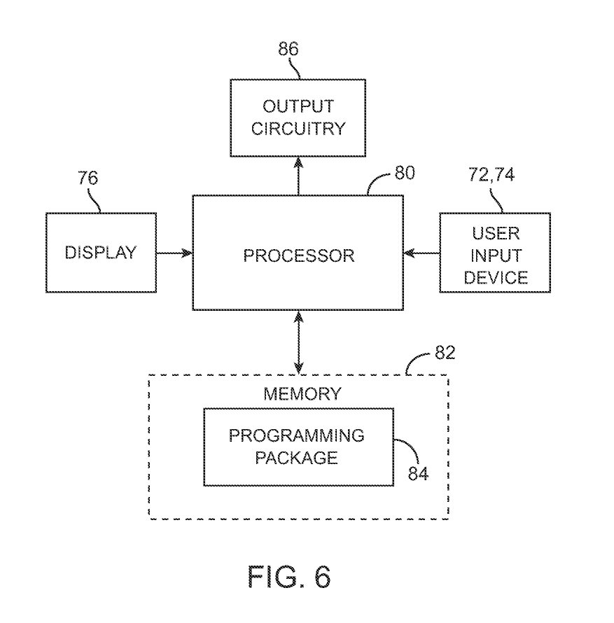

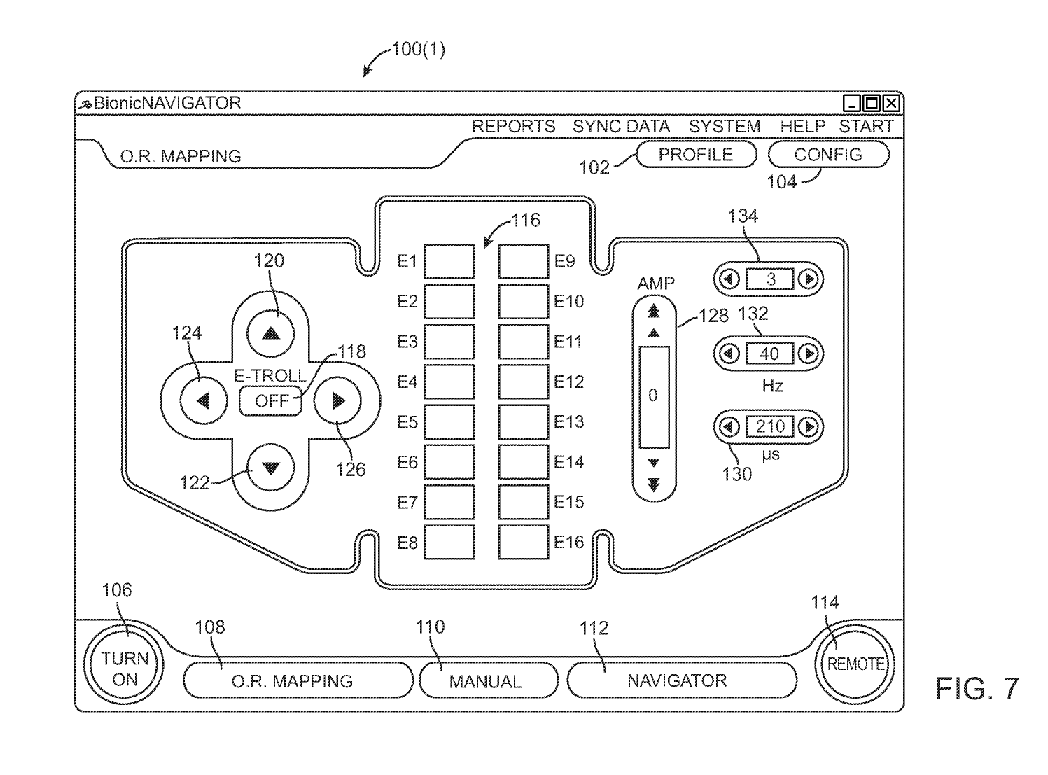

To allow the user to perform these functions, the CP 18 includes a mouse 72, a keyboard 74, and a programming display screen 76 housed in a case 78. It is to be understood that in addition to, or in lieu of, the mouse 72, other directional programming devices may be used, such as a joystick, or directional keys included as part of the keys associated with the keyboard 74. As shown in FIG. 6, the CP 18 generally includes a processor 80 (e.g., a central processor unit (CPU)) and memory 82 that stores a stimulation programming package 84, which can be executed by the processor 80 to allow the user to program the IPG 14, and RC 16. The CP 18 further includes output circuitry 86 (e.g., via the telemetry circuitry of the RC 16) for downloading stimulation parameters to the IPG 14 and RC 16 and for uploading stimulation parameters already stored in the memory 66 of the RC 16, via the telemetry circuitry 68 of the RC 16.

Referring to FIGS. 7-13, execution of the programming package 84 by the processor 80 provides a multitude of display screens 100 that can be navigated through via use of the mouse 72. As shown in each of these display screens, a profile icon 102 and a configuration icon 104 are located at the top of each of the display screens, and a power-on icon 106, OR mapping icon 108, manual icon 110, navigator icon 112, and remote icon 114 are located at the bottom of each of the display screens 100. These icons can be actuated, and in particular, clicked using the mouse 72, in order to perform various programming functions during the session.

For example, clicking on the profile icon 102 allows the user to select or enter patient profile information (e.g., name, birth date, patient identification, physician, diagnosis, and address), enter procedure information (e.g., programming/follow-up, implant trial system, implant IPG, implant IPG and lead(s), replace IPG, replace IPG and leads, replace or revise leads, explant, etc.), generate a pain map of the patient. Clicking on the configuration icon 104 allows the user to define the configuration and orientation of the neurostimulation leads 12. Clicking on the power-on icon 106 directs the IPG 14 to alternately deliver or cease delivering stimulation energy to the electrode array 26 in accordance with a defined set of stimulation parameters. Clicking on the OR mapping icon 108 allows the user to assess lead position and evaluate paresthesia coverage during surgery. Clicking on the manual icon 110 allows the user to manually select stimulation parameter sets, including fractionalized electrode combinations. Clicking on the navigator icon 112 allows the user to shift current between multiple electrode combinations to fine tune and optimize stimulation coverage for patient comfort. Clicking on the remote icon 114 allows the user to check battery status and modify patient options for the RC 16, activate stimulation programs previously stored in the RC 16 and IPG 14, and store the stimulation parameter sets created during the navigation or manual programming sessions in the RC 16 and IPG 14 as a new stimulation program.

Further details discussing the above-described CP functions are disclosed in U.S. Provisional Patent Application Ser. No. 61/080,187, entitled “System and Method for Converting Tissue Stimulation Programs in a Format Usable by an Electrical Current Steering Navigator,” which is expressly incorporated herein by reference. For purposes of brevity, only the E-troll and navigation programming functions will be discussed herein in greater detail.

Referring first to FIG. 7, clicking on the OR mapping icon 108 opens up an OR mapping screen 100(1), which as briefly discussed above, allows a clinician to assess lead position and evaluate paresthesia coverage during surgery via an Electronic Trolling (E-Troll) function. E-Troll is a quick way to sweep the electrode array by gradually moving a cathode in bipolar stimulation. To this end, the OR mapping screen 100(1) includes a graphical representation 116 of the electrode array 26 and an E-Troll control icon 118 that can be clicked to enable the E-trolling function, and up, down, left, and right current shifting arrows 120-126 to respectively move the cathode or cathodes up, down, left and right in the electrode array 26, thereby steering the electrical current, and thus, the resulting stimulation region, up, down, left, and right in the electrode array 26, in accordance with an electrical current steering pattern, which in the illustrated embodiment, is defined by a navigation table. As briefly discussed above, actuation of the power-on icon 106 in the OR mapping screen 100(1) directs the IPG 14 to alternately deliver or cease delivering stimulation energy to the electrode array 26 (corresponding to the graphical electrode representation 116) shown in FIG. 7) in accordance with the stimulation parameters generated during the E-troll function and transmitted from the CP 18 to the IPG 14 via the RC 16.

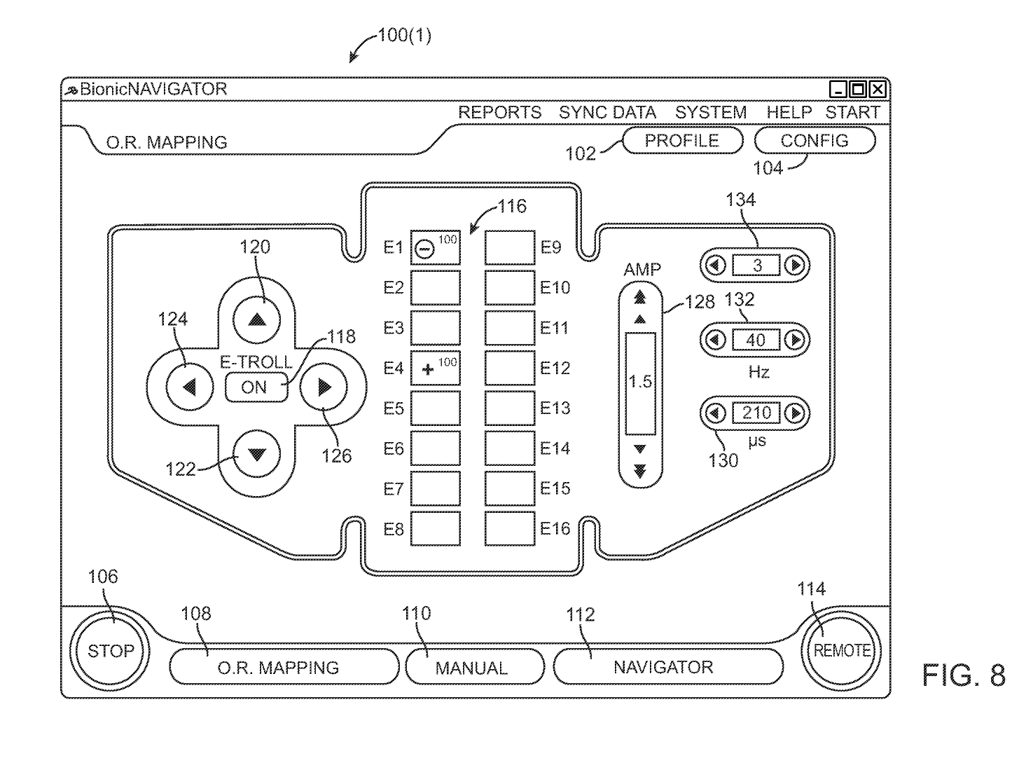

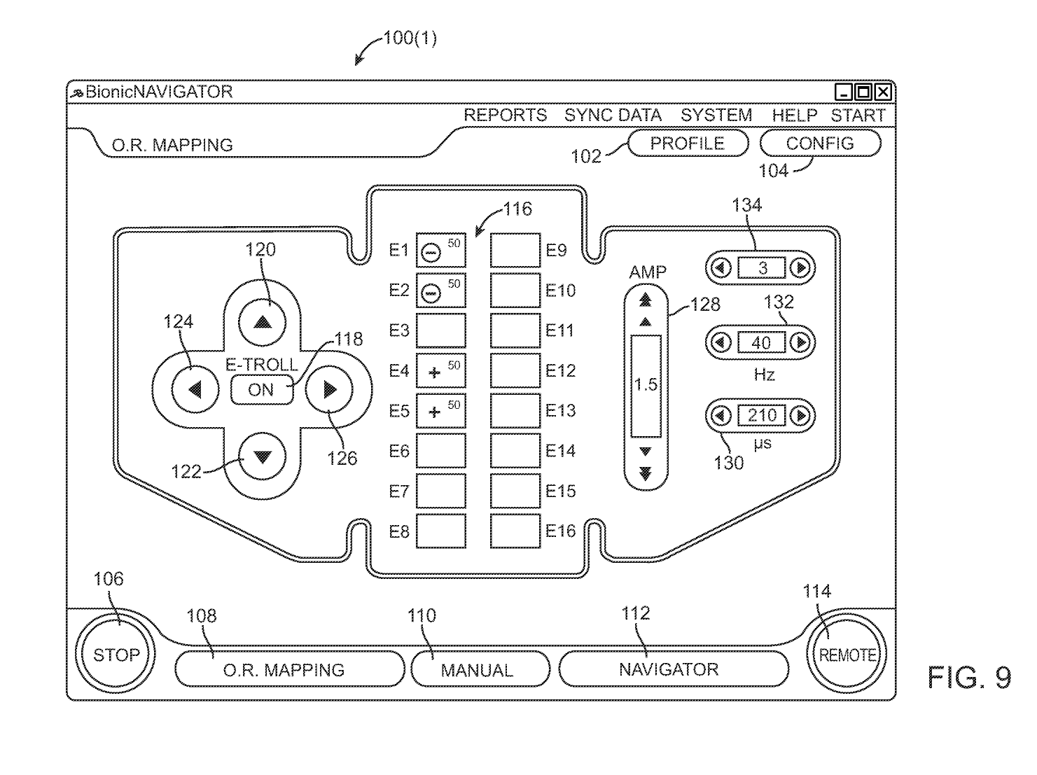

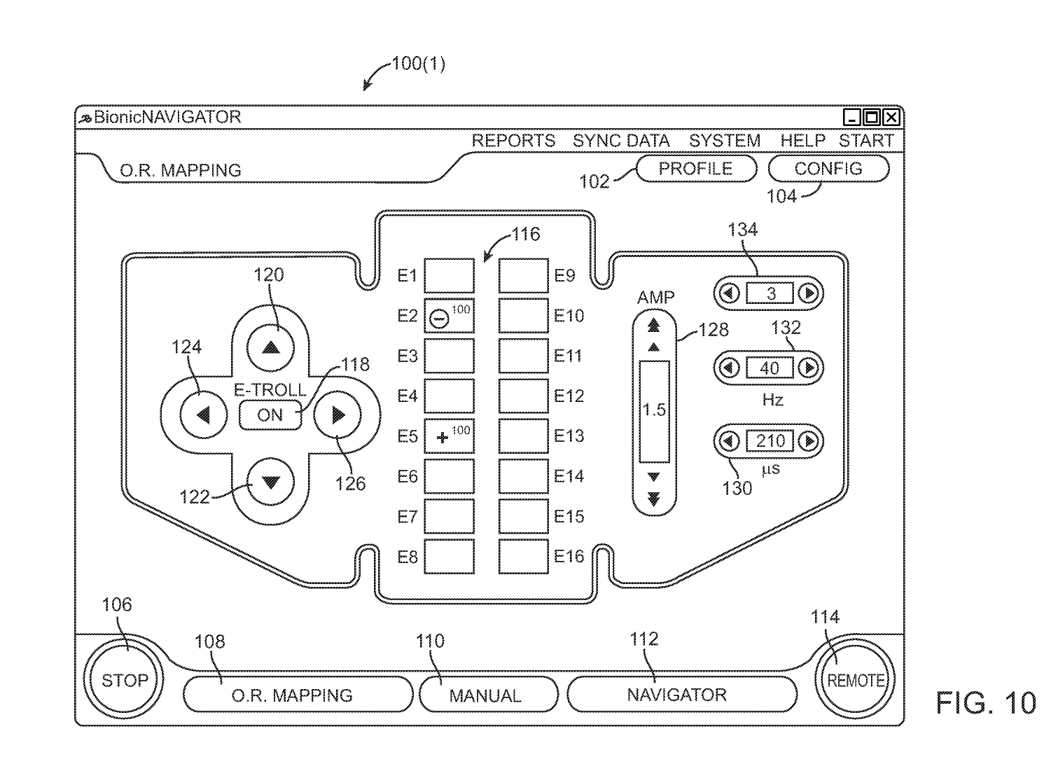

For example, as shown in FIG. 8, the E-Troll process may begin by designating electrode E1 as the sole cathode and electrode E4 as the sole anode. As there shown, electrode E1 has a fractionalized cathodic current value of 100%, and electrode E4 has a fractionalized anodic current value of 100%. If the down arrow 122 is clicked, the cathodic current is gradually shifted from electrode E1 to electrode E2, and the anodic current is gradually shifted from electrode E4 to electrode E5, which gradual shifting occurs in 10% increments. For example, as shown in FIG. 9, the electrical current is shifted, such that electrode E1 has a fractionalized cathodic current value of 50%, electrode E2 has a fractionalized cathodic current value of 50%, electrode E4 has a fractionalized anodic current value of 50%, and electrode E5 has a fractionalized anodic current value of 50%. As shown in FIG. 10, the electrical current is further shifted, such that electrode E2 has a fractionalized cathodic current value of 100%, and electrode E5 has a fractionalized anodic current value of 100%. Further clicking of the down arrow 122 shifts the cathodic current and anodic current further down the electrode array in a similar manner. Likewise, clicking the up arrow 120, left arrow 124, or right arrow 126 causes the cathodic currents and anodic currents to respectively shift up, left, and right within the electrode array in a similar manner.

In the illustrated embodiment, a navigation table, such as the one shown in Appendix A, is used to generate fractionalized electrode combinations for each neurostimulation lead 12. Because the navigation table only contains fractionalized electrode combinations for a single lead (i.e., 8 electrodes) to independently generate fractionalized electrode combinations for each neurostimulation lead 12 (one for electrodes E1-E8 and one for electrodes E9-E16), which for purposes of displaying to the OR mapping screen 100(1), can then be combined into a single fractionalized electrode combination and normalized, such that the fractionalized cathodic current for both leads 12 (i.e., the entire electrode array 26) totals 100% and the fractionalized anodic current for both leads 12 (i.e., the entire electrode array 26) totals 100%.

The cathodic and anodic currents can be shifted up and down along each neurostimulation lead 12 by stepping up and down through the fractionalized electrode combinations within the navigation table. The cathodic and anodic currents can be shifted left and right by scaling the currents on the first and second leads relative to each other. That is, to steer current from the second lead to the first lead, the fractionalized electrode combination for the second lead is scaled down, and the fractionalized electrode combination for the first lead is scaled up, and to steer current from the first lead to the second lead, the fractionalized electrode combination for the first lead is scaled down, and the fractionalized electrode combination for the second lead is scaled up.

The OR mapping screen 100(1), as shown in FIG. 10, also allows the clinician to modify the characteristics of the stimulation energy (i.e., the electrical pulse parameters) output by the IPG 14 to the electrodes during the E-troll function by adjusting each of a pulse amplitude, pulse width, or pulse rate. To this end, the OR mapping screen 100(1) includes a pulse amplitude adjustment icon 128, the top arrow of which can be clicked to incrementally increase the pulse amplitude of the stimulation energy, and the bottom arrow of which can be clicked to incrementally decrease the pulse amplitude of the stimulation energy. The OR mapping screen 100(1) further includes a pulse width adjustment icon 130, the right arrow of which can be clicked to incrementally increase the pulse width of the stimulation energy, and the left arrow of which can be clicked to incrementally decrease the pulse width of the stimulation energy. The OR mapping screen 100(1) further includes a pulse rate adjustment icon 132, the right arrow of which can be clicked to incrementally increase the pulse rate of the stimulation energy, and the left arrow of which can be clicked to incrementally decrease the pulse rate of the stimulation energy. Notably, the adjustment of the pulse amplitude, pulse width, and pulse rate will be performed globally for all of the electrodes activated as either an anode (+) or a cathode (−).

More significant to the present inventions, the OR mapping screen 100(1) further includes a stimulation transition rate adjustment icon 134, the right arrow of which can be clicked to incrementally increase the rate at which the stimulation energy is transitioned through the different stimulation parameter sets, and the left arrow of which can be clicked to incrementally decrease the rate at which the stimulation energy is transitioned through the different stimulation parameter sets. In the illustrated embodiment, the stimulation energy transition rate can be adjustment between a normalized range of 1-10. The stimulation parameter sets differ in that they may, e.g., contain different active electrode combinations, and in this case, different fractionalized electrode combinations, and/or may contain different pulse amplitudes, pulse widths, and pulse rates. That is, as any one of the arrows of the E-Troll icon 118, pulse amplitude adjustment icon 128, pulse width adjustment icon 130, pulse rate adjustment icon 132 is actuated, a series of stimulation parameter sets containing different stimulation parameter values (either fractionalized current values, pulse amplitude values, pulse width values, or pulse rate values) is generated.

The rate at which the stimulation energy is transitioned may be time-based in that it can be a function of the time interval between immediately adjacent incremental transitions of the delivered stimulation energy. For example, such a time-based rate can be defined as the inverse of the time interval between each incremental electrical current shift between cathodic electrodes or between anodic electrodes (in this case, the elapsed time between implementation of one row to the next row in the navigation table) via actuation of any of the current shifting arrows 120-126, or the inverse of the time interval between each incremental shift in the pulse rate, pulse amplitude, or pulse duration via actuation of the up/down arrows of the respective pulse amplitude adjustment icon 128, pulse width adjustment icon 130, or pulse rate adjustment icon 132. That is, if the time interval is a half-second, the stimulation energy will be transitioned two times per second. Of course, the denominator can be any unit time, e.g., minutes (in which case, the inverse of the time interval will be multiplied by 60) or hours (in which case, the inverse of the time interval will be multiplied by 3600). In any case, the time interval may range from, e.g., milliseconds to hundreds of seconds.

The rate at which the stimulation energy is transitioned may alternatively be magnitude-based in that it can be a magnitude difference between immediately adjacent stimulation parameter values within the different stimulation parameter sets. For example, such a magnitude-based rate can define the magnitude differences between each incremental electrical current shift between the cathodic electrodes or between the anodic electrodes (in this case, the difference in the fractionalized current values between implementation of one row to the next row in the navigation table) via actuation of any of the current shifting arrows 120-126 or the magnitude of the incremental shifts in the pulse amplitude, pulse width, or pulse rate via actuation of the up/down arrows of the respective pulse amplitude adjustment control icon 128, pulse width adjustment control icon 130, or pulse rate adjustment control icon 132. The magnitude-based rate at which the incremental electrical current is shifted can be adjusted by stepping through the navigation table in different manners. For example, each row of the navigation table can be stepped through, so that the magnitude-based rate is relatively small (e.g., 5% steps), and thus, the rate is relatively low, or every second row, or every third row, etc., of the navigation table can be stepped through, so that the magnitude-based rate is relatively large (e.g., 10% steps, 15% steps, etc.), and thus, the rate is relatively high. Alternatively, different navigation tables, each of which has uniform step sizes, but all of which have different step sizes relative to each other, can be used, in which case, the navigation table with the desired step size will be selected.

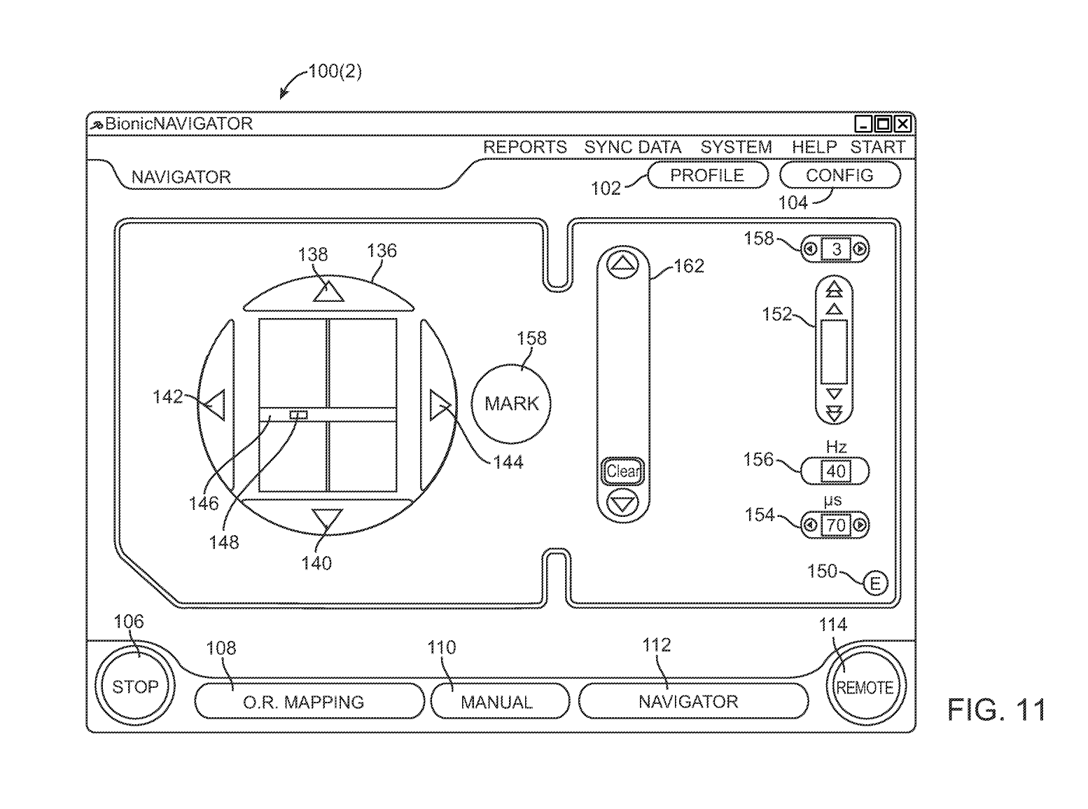

In the context of a follow-up procedure, execution of the programming package 84 may open up a navigator screen 100(2), which as briefly discussed above, allows a clinician to shift current between multiple electrode combinations to fine tune and optimize stimulation coverage for patient comfort, as shown in FIG. 11. To this end, the navigator screen 100(2) includes a navigator scope 136 that represents the stimulation region along the spinal cord relative to the electrode array that can be targeted using current shifting icons 138-144 (up, down, left, and right arrows). The navigator scope 136 has a horizontal bar 146 with a location designator (represented by a rectangular opening) 148 that indicates the current location of the stimulation region relative to the electrode array. Clicking on the up and down arrows 138, 140 displaces the horizontal bar 146, and thus the location designator 148, up and down within the navigator scope 136, and clicking on the left and right arrows 142, 144 displaces the location designator 148 left and right along the horizontal bar 146.

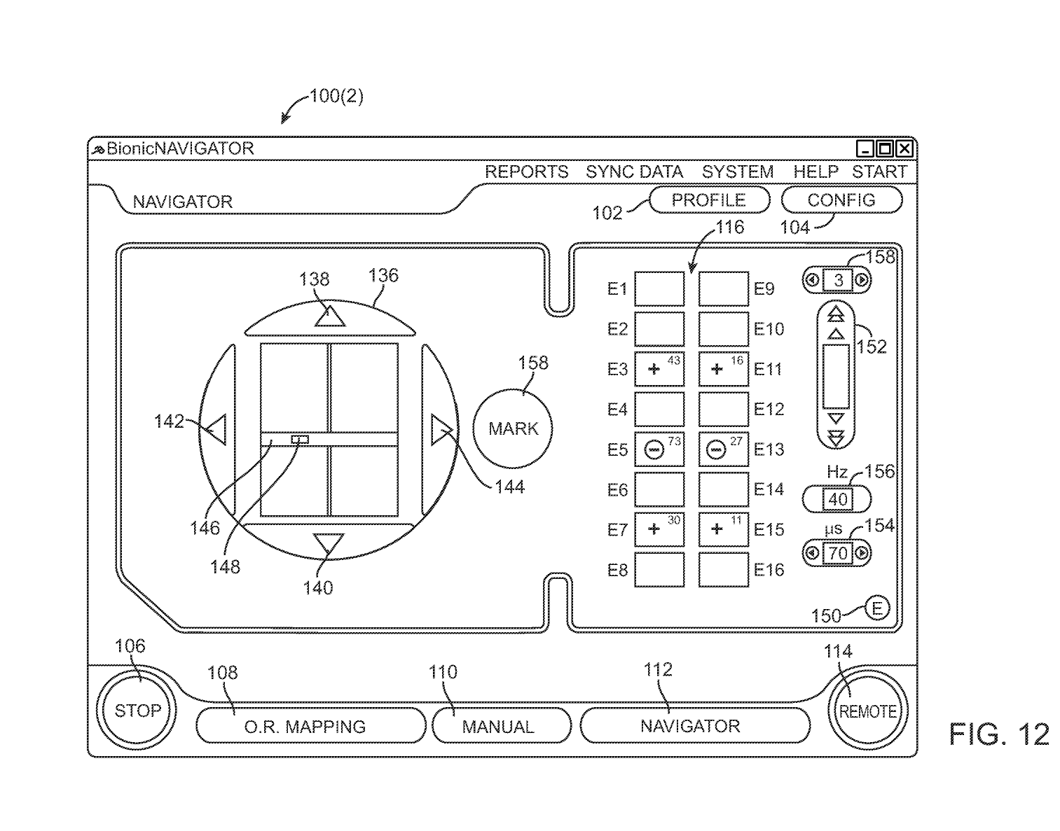

Thus, the stimulation region can be displaced upward by clicking on the up arrow 138, displaced downward by clicking on the down arrow 140, displaced to the left by clicking on the left arrow 142, and displaced to the right by clicking on the right arrow 144. As briefly discussed above, actuation of the power-on button 106 in the navigator screen 100(2) directs the IPG 14 to alternately deliver or cease delivering stimulation energy to the electrode array 26 (corresponding to the graphical electrode representation 116 shown in FIG. 12) in accordance with the stimulation parameters generated during the navigation function and transmitted from the CP 18 to the IPG 14 via the RC 16.

The navigator scope 136 displaces the stimulation region by steering the electrical current (i.e., shifting electrical current between the electrodes E1-E16) in a manner similar to that used by the E-Troll function described above to shift current between the electrodes E1-E16. Thus, clicking the up arrow 138 displaces the cathode or cathodes upward in the electrode array, thereby displacing the stimulation region upward relative the spinal cord; clicking the down arrow 140 displaces the cathode or cathodes downward in the electrode array, thereby displacing the stimulation region downward relative to the spinal cord; clicking the left arrow 142 displaces the cathode or cathodes to the left in the electrode array, thereby displacing the stimulation region to the left relative to the spinal cord; and clicking the right arrow 144 displaces the cathode or cathodes to the right in the electrode array, thereby displacing the stimulation region to the right relative to the spinal cord.

In the illustrated embodiment, a navigation table, such as the one shown in Appendix A, is used to generate fractionalized electrode combinations for each neurostimulation lead 12. Again, because the navigation table only contains fractionalized electrode combinations for a single lead (i.e., 8 electrodes), two identical navigation tables will be used to independently generate fractionalized electrode combinations for each neurostimulation lead 12 (one for electrodes E1-E8 and one for electrodes E9-E16), which for purposes of displaying to the clinician in the navigation 122, can then be combined into a single fractionalized electrode combination and normalized, such that the fractionalized cathodic current for both leads 12 (i.e., the entire electrode array 26) totals 100% and the fractionalized anodic current for both leads 12 (i.e., the entire electrode array 26) totals 100%. The cathodic and anodic currents can be shifted up and down along each neurostimulation lead 12 and shifted left and right between the neurostimulation leads 12 in the same manner described above with respect to the E-Troll function.

The navigator screen 100(2) also includes an electrode combination button 150 that can be clicked to allow clinician to view the fractionalized electrode combination that corresponds to the stimulation region identified by the location designator 148, as shown in FIG. 12. As there shown, electrodes E3, E7, E11, and E15 respectively have fractionalized cathodic current values of 43%, 30%, 16%, and 11%, and electrodes E5 and E13 respectively have anodic current values of 73% and 27% to locate the stimulation region at the location currently pointed to by the location designator 148. The navigator screen 100(2) also allows the clinician to modify the stimulation energy (i.e., the electrical pulse parameters) output by the IPG 14 by adjusting each of a pulse amplitude or a pulse width.

To this end, the navigator screen 100(2) includes a pulse amplitude adjustment icon 152, the top arrow of which can be clicked to incrementally increase the pulse amplitude of the stimulation energy, and the bottom arrow of which can be clicked to incrementally decrease the pulse amplitude of the stimulation energy. The navigator screen 100(2) further includes a pulse width adjustment icon 154 (provided only in the navigator screen 100(2) illustrated in FIG. 12), the right arrow of which can be clicked to incrementally increase the pulse width of the stimulation energy, and the left arrow of which can be clicked to incrementally decrease the pulse width of the stimulation energy. Notably, the adjustment of the pulse amplitude or pulse width will be performed globally for all of the electrodes activated as either an anode (+) or a cathode (−). While the navigator screen 100(2) does not include a pulse rate adjustment icon, it does include a pulse rate display 156 that provides the default pulse rate for the system to the clinician.

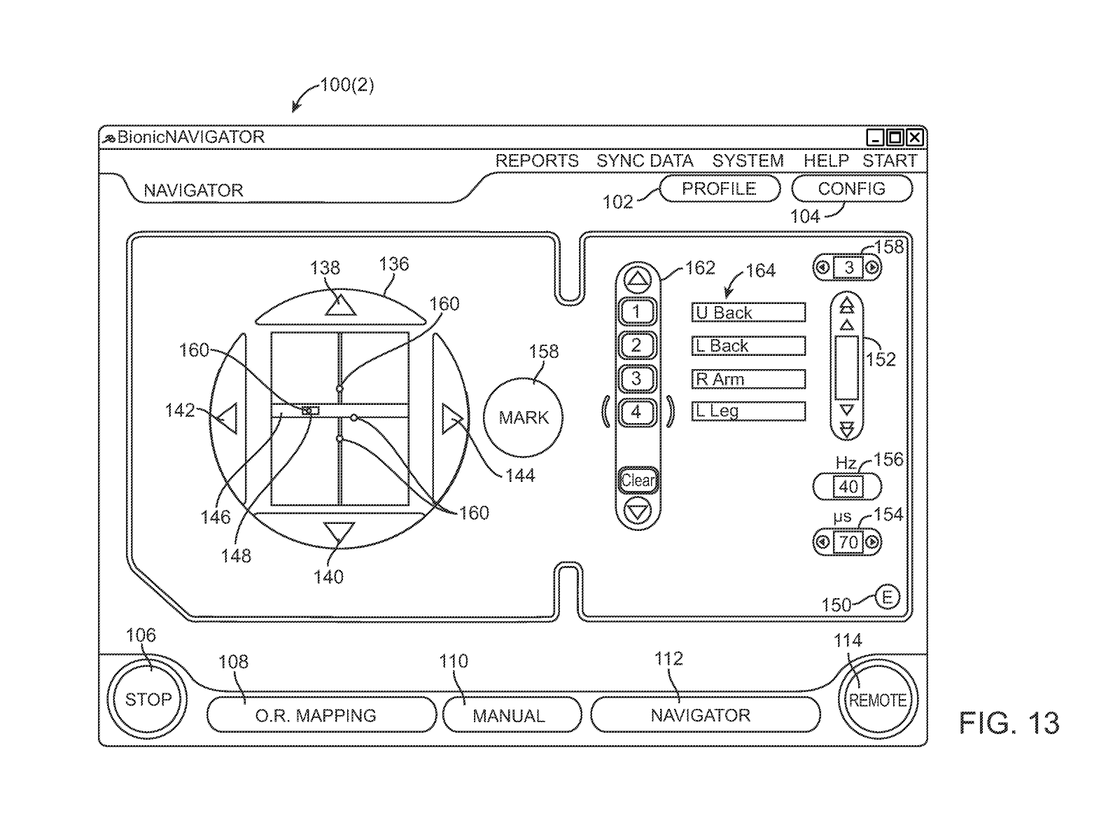

The navigator screen 100(2) has a mark button 158 that can be clicked to mark points 160 (shown in FIG. 13) where coverage is preferred for the target area; that is, the area that the location designator 156 currently points to when the mark button 156 is clicked will be marked. Each mark 160 is a set of stimulation parameters (including fractionalized electrode configuration, pulse amplitude, pulse width, and pulse rate) that corresponds to the location or area of the stimulation region. As shown in FIG. 13, the navigator screen 100(2) includes a mark list 162 that includes numbered designators corresponding to all of the marks 160 generated by the navigator scope 136 and an area designator 164 that can be filled in by the clinician to associate an area of paresthesia for each mark 160. As shown in FIG. 13, four marks 160 have been generated, with the first mark being identified as causing paresthesia in the upper back of the patient, the second mark being identified as causing paresthesia in the lower back of the patient, the third mark being identified as causing paresthesia in the right arm of the patient, and the fourth mark being identified as causing paresthesia in the left leg of the patient. Notably, any one of the numbered designated within the mark list 162 can be clicked to center the area designator 164 on the corresponding mark 160 in the navigation scope 136.

Like the OR mapping screen 100, the navigator screen 100(2) further includes a stimulation transition rate adjustment control icon 166, the right arrow of which can be clicked to incrementally increase the rate at which the stimulation energy is transitioned through the different stimulation parameter sets, and the left arrow of which can be clicked to incrementally decrease the rate at which the stimulation energy is transitioned through the different stimulation parameter sets. Operation of the stimulation transition rate adjustment control icon 166 operates in the same manner as that described above with respect to the stimulation transition adjustment control icon 134 in the OR mapping screen 100(1).

It can be appreciated that, by using the stimulation transition rate adjustment control icon 134 in the OR mapping screen 100(1) or the stimulation transition rate adjustment control icon 166 in the navigator screen 100(2), the user can adjust the rate at which the stimulation energy is transitioned based on feedback from the patient. In particular, the stimulation energy applied to the patient can be incrementally transitioned through a first series of different stimulation parameter sets at a defined rate in response to a single user actuation of the stimulation transition rate adjustment control mechanism (e.g., any of the current shifting arrows 120-126 of the E-Troll control icon 118, up/down arrows of the pulse amplitude adjustment icon 128, pulse duration adjustment icon 130, or pulse rate adjustment icon 132 in the OR mapping screen 100(1) or any of the current shifting icons 138-144 of the navigator scope 136, or up/down arrows of the pulse amplitude adjustment icon 152 or pulse width adjustment icon 154).

If the patient cannot feel the changes in the stimulation energy transitions, or the user otherwise needs to reduce the stimulation energy transition rate, e.g., to allow more time to manually adjust stimulation parameters other than those being automatically transitioned (e.g., manually changing the pulse amplitude, pulse width, or pulse rate between transitions in the electrode combination fractionalizations) or if the currently tested stimulation parameter sets are close to the optimum stimulation parameter set, the user can click on the left arrow of the stimulation transition adjustment control icon 134 in the OR mapping screen 100(1) or the stimulation transition adjustment control icon 166 in the navigator screen 100(2) to decrease the stimulation transition rate. Thus, the stimulation energy can be incrementally transitioned in a manner that avoids drastic changes in the stimulation.

If the user needs to increase the stimulation energy transition rate, e.g., if the stimulation energy transitions are too slow or the currently tested stimulation parameter sets are far from the optimum stimulation parameter set, the user can click on the right arrow of the stimulation transition adjustment control icon 134 in the OR mapping screen 100(1) or the stimulation transition adjustment control icon 166 in the navigator screen 100(2) to increase the stimulation transition rate until the patient is capable of feeling the changes in the stimulation energy transitions.

Once the stimulation energy transition rate is properly adjusted (either by decreasing it or increasing it), the stimulation energy applied to the patient can be incrementally transitioned through a second series of different stimulation parameter sets at a defined rate in response to another single user actuation of the stimulation transition rate adjustment control mechanism. If optimum or otherwise effective to provide the necessary therapy to the patient, at least one of the second series of stimulation parameter sets can be programmed in the IPG 14 (or alternatively, the ETS 20) when operating the CP 18 in the navigator screen 100(2).

Although the foregoing techniques have been described as being implemented in the CP 18, it should be noted that this technique may be alternatively or additionally implemented in the RC 16. Furthermore, although the control mechanisms for generating stimulation parameter sets and adjusting the rate at which the stimulation parameter sets are transitioned have been described as computer icons that can be clicked using a mouse, other types of control mechanisms, such as a button, dial, slider bar, etc., can be used.

Although particular embodiments of the present inventions have been shown and described, it will be understood that it is not intended to limit the present inventions to the preferred embodiments, and it will be obvious to those skilled in the art that various changes and modifications may be made without departing from the spirit and scope of the present inventions. Thus, the present inventions are intended to cover alternatives, modifications, and equivalents, which may be included within the spirit and scope of the present inventions as defined by the claims.

What is claimed is:

1. A neurostimulation system, comprising:
a neurostimulator configured for delivering electrical stimulation energy to a tissue region in accordance with different stimulation parameter sets; and
an external control device configured for, in response to a single user actuation of a control mechanism, instructing the neurostimulator to incrementally transition the delivered electrical stimulation energy through the different stimulation parameter sets at a user-adjustable rate.

2. The neurostimulation system of claim 1, wherein the different parameter sets respectively contain different active electrode combinations.

3. The neurostimulation system of claim 2, wherein the electrode combinations are fractionalized electrode combinations.

4. The neurostimulation system of claim 1, wherein the different parameter sets respectively contain different values for at least one of a pulse amplitude, a pulse width, and a pulse rate.

5. The neurostimulation system of claim 1, wherein the user-adjustable rate is a function of a time interval between immediately adjacent incremental transitions of the delivered electrical stimulation energy.

6. The neurostimulation system of claim 1, wherein the user-adjustable rate is a function of a magnitude difference between immediately adjacent stimulation parameter values within the different stimulation parameter sets.

7. The neurostimulation system of claim 1, wherein the external control device is further configured for programming at least one of the stimulation parameter sets into the neurostimulator.

8. The neurostimulation system of claim 1, wherein the control mechanism is a computer icon.

9. A neurostimulation control system for a neurostimulator that conveys electrical stimulation energy to a tissue region in accordance with different electrical stimulation parameter sets, comprising:
a control mechanism configured for being actuated by a user;
at least one processor configured for generating the different stimulation parameter sets in response to a single user actuation of the control mechanism;
telemetry circuitry configured for transmitting the different stimulation parameters to the neurostimulator, such that the neurostimulator incrementally transitions the delivered electrical stimulation energy through the different stimulation parameter sets at a defined rate; and
a user interface configured for allowing a user to adjust the defined rate.

10. The neurostimulation control system of claim 9, wherein the different parameter sets respectively contain different active electrode combinations.

11. The neurostimulation control system of claim 10, wherein the electrode combinations are fractionalized electrode combinations.

12. The neurostimulation control system of claim 9, wherein the different parameter sets respectively contain different values for at least one of a pulse amplitude, a pulse width, and a pulse rate.

13. The neurostimulation control system of claim 9, wherein the defined rate is a function of the inverse of a time interval between immediately adjacent incremental transitions of the delivered electrical stimulation energy.

14. The neurostimulation control system of claim 9, wherein the defined rate is a function of a magnitude difference between immediately adjacent stimulation parameter values within the different stimulation parameter sets.

15. The neurostimulation control system of claim 9, wherein the control mechanism is a computer icon.

16. A method of providing therapy to a patient, comprising:
delivering electrical stimulation energy to a tissue region in accordance with different stimulation parameter sets;
incrementally transitioning the delivered electrical stimulation energy through a first series of the different stimulation parameter sets at a user-defined rate in response to a single user actuation of a control mechanism;
adjusting the user-defined rate; and
incrementally transitioning the delivered electrical stimulation energy through a second series of the different stimulation parameter sets at the adjusted rate in response to a single user actuation of the control mechanism.

17. The method of claim 16, wherein the different parameter sets respectively contain different active electrode combinations.

18. The method of claim 17, wherein the electrode combinations are fractionalized electrode combinations.

19. The method of claim 16, wherein the different parameter sets respectively contain different values for at least one of a pulse amplitude, a pulse width, and a pulse rate.

20. The method of claim 16, wherein the defined rate is a function of the inverse of a time interval between immediately adjacent incremental transitions of the delivered electrical stimulation energy.

21. The method of claim 16, wherein the defined rate is a function of a magnitude difference between immediately adjacent stimulation parameter values within the different stimulation parameter sets.

22. The method of claim 16, further comprising programming at least one of the second series of stimulation parameter sets into the memory of a neurostimulation device.

23. The method of claim 16, wherein the control mechanism is a computer icon.

24. The method of claim 16, wherein the control mechanism is a directional device.
